(19) United States
(12) Patent Application Publication
(10) Pub. No.: US 2020/0260700 A1
(43) Pub. Date: Aug. 20, 2020

(54) Transgenic Insect and Use of Same in Methods for Testing Natural or Synthetic Substances

(71) Applicants: Robert Bosch Gesellschaft für Medizinische Forschung mbH, Stuttgart (DE); Universite Nice Sophia Antipolis, Nice Cedex 2 (FR)

(72) Inventors: Elke Schaeffeler, Stuttgart (DE); Bernard Moussian, Tübingen (DE); Yiwen Wang, Dusslingen (DE); Matthias Schwab, Stuttgart (DE); Anne Nies, Stuttgart (DE); Bernhard Spengler, Linden (DE)

(21) Appl. No.: 16/756,589

(22) PCT Filed: Nov. 6, 2018
(86) PCT No.: PCT/EP2018/080307

§ 371 Date: Apr. 16, 2020

(30) Foreign Application Priority Data
Nov. 9, 2017 (DE) 10 2017 219 981.4

(51) Int. Cl. A01K 67/033; C07K 14/705

(52) U.S. Cl. CPC A01K 67/0339; C07K 14/705; A01K 2267/01; A01K 2227/706; A01K 2217/05

(57) ABSTRACT

A transgenic insect includes a genome which has at least one first exogenic DNA sequence, which is coded for a human membrane transporter protein. The expression of the first exogenic DNA sequence leads to a functional human membrane transporter protein in the transgenic insect.

The present invention relates to a transgenic insect, the genome of which comprises an exogenous DNA sequence. The invention further relates to the use of a vector for generating a transgenic insect and to methods for testing natural or synthetic substances using the transgenic insect.

### PRIOR ART

In drug development, information about clinical factors such as the blood and tissue concentration of drugs and active pharmacological ingredients and also the metabolites thereof is of utmost importance. This knowledge substantially contributes to being able to better understand the therapeutic efficacy and the occurrence of adverse effects (Giacomini et al., Nat. Rev. Drug Discov. 2010).

In this connection, an important role is played by membrane transporter proteins (or membranous transporter proteins) in particular, since they are capable of having a crucial influence on the absorption, distribution and elimination of, inter alia, active ingredients. Said transporter proteins are integral constituents of the lipid bilayer of biological membranes. They regulate or control the transport of numerous substances into the cells (influx) or out of them (efflux). Substance exchange through said membrane transporter proteins can either be facilitated in a passive manner or be effected in an active manner.

For example, membrane transporters are important determinants of pharmacokinetics, i.e., the uptake of an active ingredient (absorption), its distribution in the body (distribution), its biochemical conversion and degradation (metabolization) and also its elimination (excretion). It is known for example that, among the top 200 prescription drugs in the USA that are eliminated via the kidneys, about 41% interact with membrane transporters (Morrissey et al., Annu. Rev. Pharmacol. Toxicol. 2013). In particular, secretory membrane transporters are important determinants of the disposition of foreign substances (xenobiotics), including many prescription drugs.

Because of this, both the EMA (European Medicines Agency) and the FDA (Food and Drug Administration) require the testing of at present eleven relevant human membrane transporters for possible interactions with novel or newly authorizable drugs (Guideline on the investigation of drug interactions, European Medicines Agency, 2012; FDA draft guidance. Drug interaction studies—study design, data analysis, implications for dosing, and labeling recommendations, 2012). These tests are an essential and mandatory part of the preclinical drug development process. It is, for example, possible with in vitro systems to predict as far as possible the active ingredient substrate profile of membrane transporter proteins. Mammalian cell lines which recombinantly express human membrane transporter proteins are, for example, suitable for in vitro models. In culture, they form a cellular monolayer, on which it is possible to study the permeability and the transport of active ingredients. A further established test is the mouse model. However, this is an in vivo test system.

The test systems currently used have numerous disadvantages, however. The systems are time-consuming and cost-intensive. In this connection, providing the particular system is, in particular, highly complex. Furthermore, high operation and maintenance costs arise, for example for the large number of mice which must be bred beforehand with genetically modified membrane transporters. The ethical concerns, which must always be considered, are not to be neglected either when it is necessary to carry out a multiplicity of mouse experiments.

For this reason, there is an urgent need for rapid and inexpensive test systems for preclinically clarifying the effect of human membrane transporter proteins in the drug development process. The system should, in this connection, be able to overcome the disadvantages prevailing in the prior art.

### DISCLOSURE OF THE INVENTION

Against this background, it is an object of the present invention to provide a model or test system, by means of which the aforementioned disadvantages from the prior art can be reduced or completely avoided.

According to the invention, these and other goals are achieved by a transgenic insect, the genome of which comprises at least one first exogenous DNA sequence encoding a human membrane transporter protein, the expression of the first exogenous DNA sequence leading to a functional human membrane transporter protein in the transgenic insect.

Furthermore, the goal is achieved by the use of a vector for generating transgenic insects, preferably transgenic Drosophila, the vector comprising a first DNA sequence encoding a human membrane transporter protein, the human membrane transporter protein being especially an uptake transporter protein or an efflux transporter protein, especially preferably selected from the group consisting of: OCT1 (gene symbol: SLC22A1), OCT2 (SLC22A2), OATP1B1 (SLCO1B1), OATP1B3 (SLCO1B3), OAT1 (SLC22A6), OAT3 (SLC22A8), MDR1 (ABCB1), BSEP (ABCB11), BCRP (ABCG2), MATE1 (SLC47A1), MATE2 (ALC47A2), or a genetic variant of these transporters.

A “transgenic insect” is understood here and in general to mean an insect, the genetic material of which has been specifically modified by means of genetic methods. In this connection, a transgene can be introduced into the insect. According to the invention, what is suitable as a transgenic insect is preferably a model organism, for example Drosophila. To obtain such a transgenic insect, vectors containing a specific foreign gene are introduced into a fertilized egg. The progenies obtained are transfected with a certain probability and can be subsequently screened for the foreign gene. The transgenic insect can also be obtained by specific crossing. Furthermore, the transgenic insect can also be obtained by introduction of a transgene into the insect and subsequent crossing with a further insect which may be genetically modified. According to the invention, the transgenic insect can be obtained by all methods known in the prior art for modifying genes in organisms.

According to the invention, the foreign gene or transgene is a first exogenous DNA sequence encoding a human membrane transporter protein.

The “human membrane transporter proteins” are those which naturally occur in the human genome. For example, what are important for the absorption of substances in humans are especially the uptake transporters in the gastrointestinal tract. Besides the nutrient-specialized amino acid, peptide, glucose, nucleotide or fatty acid transporters, these also include the organic anion transporter polypeptides (OATP) and the organic cation transporter proteins (OCT). By contrast, efflux transporters in the epithelium of the gastrointestinal tract such as the ABC proteins (ATP-binding cassette proteins) lower the absorption and thus the oral bioavailability of the active ingredients which bind to the transporters and are transported out of the cells by said transporters. What are crucial for elimination through the liver are especially membrane transporter proteins such as organic anion transporter polypeptides (OATP), organic cation transporter proteins (OCT) and also the P-glycoprotein (MDR1 P-gp), the bile salt efflux transporter (BSEP) and the breast cancer resistance protein (BCRP). What are crucial for elimination through the kidneys are especially transporter proteins such as organic anion transporters (OAT), organic cation transporter proteins (OCT) and also multidrug resistance and toxin extrusion proteins (MATE). In other cell membranes too, particularly at biological barriers such as the blood-brain barrier or the blood-placenta barrier, specific transporter proteins are present for regulating substance exchange.

Accordingly, it is preferred in one embodiment of the present invention when the human membrane transporter protein is a human uptake transporter protein or a human efflux transporter protein, and especially a membrane transporter protein selected from the group consisting of: OCT1 (gene symbol: SLC22A1), OCT2 (SLC22A2), OATP1B1 (SLCO1B1), OATP1B3 (SLCO1B3), OAT1 (SLC22A6), OAT3 (SCL22A8), MDR1 (ABCB1), BSEP (ABCB11), BCRP (ABCG2), MATE1 (SLC47A1), MATE2 (SLC47A2) or a genetic variant of these transporters. As already mentioned above, said uptake and efflux transporter proteins are used as part of preclinical drug studies with respect to their interaction with the particular substances to be studied. The proteins are organic cation transporter proteins (OCT), organic anion transporter polypeptides (OATP), organic anion transporters (OAT), P-glycoprotein, also known as multidrug resistance protein (MDR1), bile salt efflux transporter (BSEP), breast cancer resistance protein (BCRP) and also multidrug and toxin extrusion proteins (MATE). The sequences of these and further membrane transporter protein genes are known, and accessible under www.genenames.org for example.

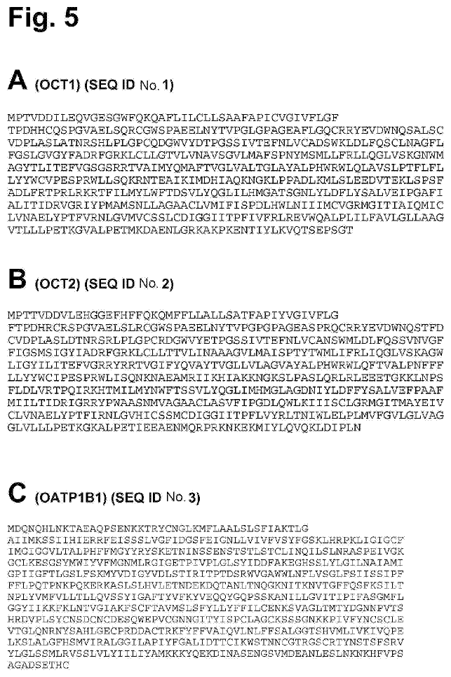

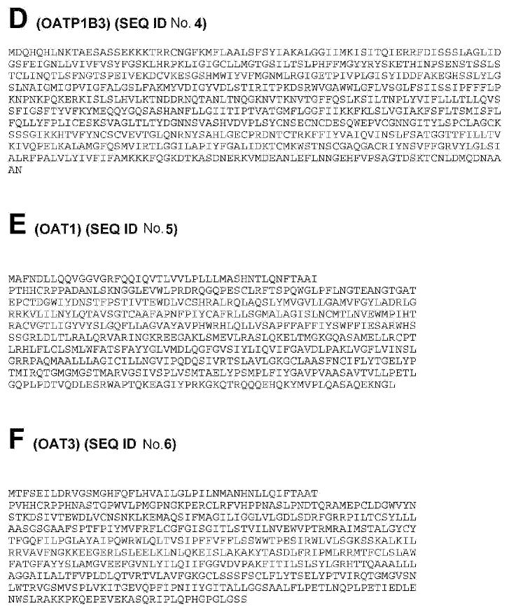

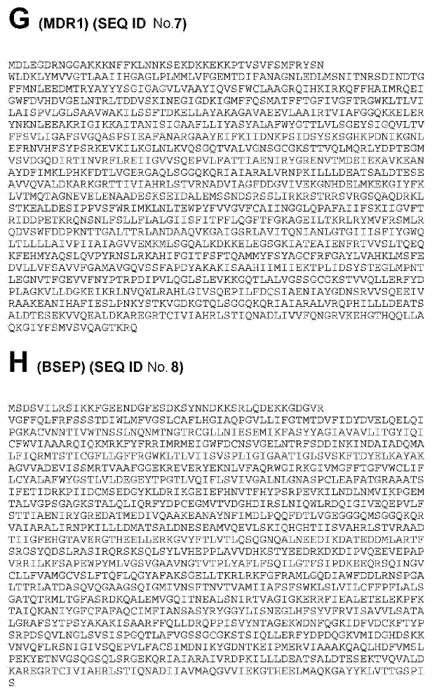

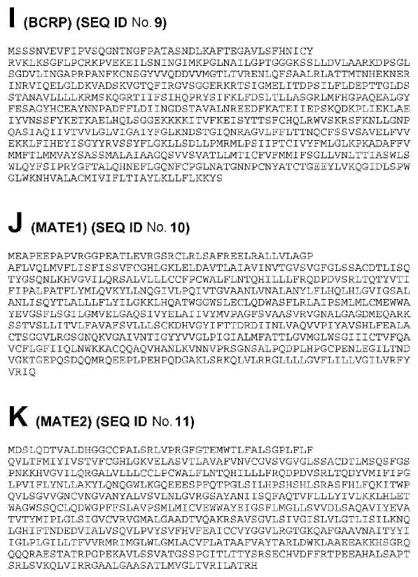

For example, it is preferred in one embodiment of the present invention when a membrane transporter protein comprising an amino acid sequence selected from one of the sequences from FIG. 5, or from a DNA sequence encoding said sequences, is used. FIG. 5 shows, by way of example, amino acid sequences of membrane transporter proteins which may, in the context of the present invention, be suitable for the described purposes and methods.

In the context of the present invention, a “DNA sequence encoding a human membrane transport protein” is understood here to mean any double-stranded nucleic acid molecule encoding a human membrane transport protein.

According to the invention and on the basis of the knowledge and experience of an average person skilled in the art, a “genetic variant” of a human membrane transporter protein is a natural protein which differs in its amino acid sequence from the wild-type or reference sequence and is, for example, characterized by the substitution, the deletion or the insertion of one amino acid or multiple amino acids. However, a genetic variant of a human membrane transporter protein preferably continues to have the same function as the naturally occurring human membrane transporter protein having the wild-type or reference sequence.

The genetic variants can be those variants which occur naturally or are specifically introduced. The variants can already be on the DNA to be transcribed and thus lead to a variant of the listed proteins.

In one embodiment, it is preferred when the transgenic insect comprises at least 1, 2, 3, 4, 5, 6, 7, 8, 9, 10 or more exogenous DNA sequences encoding a human membrane transporter protein in each case, the expression of the exogenous DNA sequences leading to functional human membrane transporter proteins in the transgenic insect.

In this connection, an “exogenous DNA sequence” is understood to mean any DNA sequence which does not occur naturally in the insect in question, i.e., without genetic modification of the insect, and which is transferred from another organism, in this case human, into the genome of the insect, where it is stably incorporated. Accordingly, “comprise an exogenous DNA sequence” means that the DNA sequence from another organism (in this case: human) has been incorporated in the insect, i.e., in the genome of the insect, for the expression thereof and is present there in a stably integrated state.

In a preferred embodiment, the expression of the first exogenous DNA sequence of the human membrane transporter protein in the insect is tissue-specific, especially salivary gland tissue-specific, intestinal system tissue-specific, tracheal tissue-specific or Malpighian tissue-specific.

According to the invention, the expression of the first exogenous DNA sequence of the human membrane transporter protein in the transgenic insects is tissue-specific. This allows more accurate studying of the human membrane transporters. If the expression is only effected in one tissue, said tissue can be studied separately, meaning that interference factors such as overexpression in the entire insect can be avoided.

Furthermore, this embodiment offers the advantage that the expressed human membrane transporter protein can be studied in a tissue-specific manner. This allows, for example, the study of membrane transporter proteins in the salivary glands, in the intestine, in the tracheae or in the Malpighian tissue. Further arbitrary combinations which are non-tissue-specific are also possible.

Salivary gland tissue specificity in particular is advantageous, since the salivary gland target tissue is nonessential in embryonic and early larval stages, meaning that a tissue manipulation, including the expression of human proteins and the functional analyses thereof, cannot be associated with influencing of the fitness and viability of the embryos. Furthermore, the epithelial cells of the salivary glands are polar and therefore resemble the human liver or kidney cells, which mainly express clinically relevant human membrane transporters, for example OCT1, OCT2, OATP1B1, OATP1B3 and MDR1.

In a preferred embodiment, the transgenic insect is a fruit fly, especially one belonging to the genus Drosophila.

Drosophila is a genus from the family of the fruit flies (Drosophilidae). Said genus also includes the fruit fly Drosophila melanogaster, which is known as a common model organism in genetics. A particular advantage of this model organism is that this fly species can be bred very easily and cheaply. In genetic research, preference is given to using Drosophila as a research object because it has a short succession of generations of only about 9 to 14 days, up to 400 progenies descend from one generation, each individual has only four chromosome pairs, and because the species shows many easily discernible gene mutations. In comparison, model systems based on a mammal are distinctly more expensive and complex.

In a preferred embodiment, the expression of the first exogenous DNA sequence encoding a human membrane transporter protein is under the control of a tissue-specific GAL4/UAS expression system, especially under the control of a salivary gland tissue-specific, intestinal system tissue-specific, tracheal tissue-specific or Malpighian tissue-specific GAL4/UAS expression system. Furthermore, it is also possible to express the first exogenous DNA sequence with the aid of the Q system (Potter et al., Cell 2010).

What is provided by the GAL4/UAS system is a genetic tool which allows the expression of foreign genes in specifically selected cells or tissues. Two modules are used for this purpose. Firstly, the GAL4 gene activator of the baker's yeast (Saccharomyces cerevisiae) is cloned under the control of specific regulatory elements. GAL4 encodes a yeast-specific transcription factor, the expression of which is under the control of a cell- or tissue-specific promoter from the fly. The other module contains the gene which encodes a human membrane transporter protein and it is under the control of UAS elements (upstream activating sequences), the target sequences of the GAL4 gene regulator. The specific binding of the GAL4 to the so-called UAS ensures the activation of the downstream target gene encoding a human membrane transporter protein. By crossing GAL4 lines and those lines bearing the target gene, it is possible to produce such systems.

The GAL4/UAS system is advantageously applicable in insects, especially in Drosophila. According to the invention, GAL4 lines which express in a tissue-specific manner can be used to obtain a transgenic insect according to this embodiment. The GAL4 is, then, only expressed in the specific tissues.

In a further aspect of the invention, the genome of the transgenic insect comprises at least one second exogenous DNA sequence encoding a fluorescent protein, especially a fluorescent protein selected from GFP, CFP, YFP, mCherry, dsRed or variants thereof.

In this embodiment, it is advantageously possible, as a result of the fluorescence of the particular proteins which can be specifically expressed in the particular tissues, to be able to directly observe the spatial and temporal distribution in living organisms. For example, the fluorescent protein can be used as a marker for a protein, for example the human membrane transporter protein, or for visualizing the tissue in which the human membrane transporter protein is expressed. Here, the first exogenous DNA sequence can be connected to the second exogenous DNA sequence. The proteins and hence the biological processes can thus be visualized in vivo, for example by means of fluorescence microscopy or confocal laser scanning microscopy.

Suitable as fluorescent proteins are, inter alia, the following UV proteins such as, for example, Sirius, Sandercyanin, shBFP-N158S/L173I; blue proteins such as, for example, Azurite, EBFP2, mKalamal, mTagBFP2, TagBFP, shBFP; cyan proteins such as, for example, ECFP, Cerulean, mCerulean3, SCFP3A, CyPet, mTurquoise, mTurquoise2, TagCFP, mTFP1, monomeric Midoriishi Cyan, Aquamarine; green proteins such as, for example, TurboGFP, TagGFP2, mUKG, Superfolder GFP, Emerald, EGFP, monomeric Azami Green, mWasabi, Clover, mNeonGreen, NowGFP, mClover3; yellow proteins such as, for example, TagYFP, EYFP, Topaz, Venus, SYFP2, Citrine, Ypet, lanRFP-ΔS83, mPapaya1, mCyRFP1; orange proteins such as, for example, monomeric Kusabira Orange, mOrange, mOrange2, mKOκ, mKO2; red proteins such as, for example, TagRFP, TagRFP-T, mRuby, mRuby2, mRuby3, mTangerine, mApple, mStrawberry, FusionRed, mCherry, mNectarine, mScarlet, mScarlet-I; dark red proteins such as, for example, mKate2, HcRed-Tandem, mPlum, mRaspberry, mNeptune, NirFP, TagRFP657, TagRFP675, mCardinal, mStable, mMaroon1, mGarnet2; near IR proteins such as, for example, iFP1.4, iRFP713 (iRFP), iRFP670, iRFP682, iRFP702, iRFP720, iFP2.0, mIFP, TDsmURFP, miRFP670; sapphire-type proteins such as, for example, Sapphire, T-Sapphire, mAmetrine; or long Stokes shift proteins such as, for example, mKeima, mBeRFP, LSS-mKate1, LSS-mKate2, LSSmOrange, CyOFP1, Sandercyanin.

Fluorescent protein variants can, for example, be the redox-sensitive variants of the GFP; possibilities here are RoGFP, rxYFP or HyPer. Furthermore, voltage-dependent GFP variants such as PROPS or VSFP are known. According to the invention, those proteins which are of natural origin, but are present in a modified state because of mutations, are also understood as fluorescent protein variant.

In a further aspect of the invention, said invention provides a method for generating a transgenic insect, the method comprising the following steps:
a) subcloning a first exogenous DNA sequence encoding a human membrane transporter protein into an expression vector to obtain a vector comprising the first exogenous DNA sequence;
b) introducing the vector obtained in step a) into an insect to obtain a stable strain of a transgenic precursor insect; and
c) crossing the transgenic precursor insect with an insect comprising an expression system matched with the expression vector to obtain the transgenic insect.

In this embodiment of the invention, a transgenic insect can be produced rapidly and reliably. In the subcloning, the first exogenous DNA sequence is introduced into another DNA sequence, the expression vector. To this end, the exogenous DNA sequence and the expression vector can be specifically cut with the aid of restriction enzymes in order to obtain so-called complementary “sticky ends” or “smooth” “blunt ends”, which can be subsequently ligated to one another, with the result that the desired expression vector is obtained. In this process, the ligation can be carried out with the aid of DNA ligases. The introduction of the first exogenous DNA sequence into the expression vector can also be effected by other methods, for example the In-Fusion Cloning System (Takara Bio USA Inc.).

According to the invention, it is possible to use plasmids, cosmids or YACs and modified viruses as expression vectors; preference is given to using a plasmid vector.

For example, a UAS-suitable vector can be used. This allows the use of the GAL4/UAS system in the generation of a transgenic insect. Furthermore, the vector can comprise a gene sequence encoding a certain phenotype. As a result, it is advantageously possible to carry out a later selection of transgenic precursor insects on the basis of the phenotype. In this case, the distinct phenotype serves as a selection marker. Mutations which can lead to an altered phenotype affect, for example, eye color, body color, eye morphology, the morphology of extremities, including wings, body setae, body colors, etc.

In a preferred embodiment, the human membrane transporter gene can be introduced into the attP attachment site in the expression vector, with the result that, for example, a pUASTattB expression vector is obtained.

In the next step, step b), the vector produced is introduced into an insect. In this process, the vector can be microinjected into an insect embryo. The stable strain of a transgenic precursor insect can be subsequently obtained with a certain probability. If the expression vector comprises a selection marker, the transgenic precursor insects can be identified on the basis thereof and sorted out accordingly. The selection marker used can be, for example, eye color or body color.

In the last step of the method for generating a transgenic insect, the precursor insect is crossed with a further insect. The two insects differ in their genome. Whereas the precursor insect already contains the gene for the human membrane transporter protein, the other insect does not comprise said gene. Preferably, the last-mentioned insect is a tissue-specific line, the expression system of which is matched with that of the precursor insect.

Crossing which has proceeded correctly can be confirmed by reverse transcriptase PCR as a result of expression in the progenies. Membrane localization of the human transporter proteins in the specific tissues can be verified by standard immunostaining techniques using validated primary antibodies. For example, immunofluorescence images can be obtained by confocal laser scanning microscopy.

In a further embodiment of the method, the expression system is GAL4/UAS.

Advantageously, the expression vector in which the first exogenous DNA sequence encoding a human membrane transporter protein is subcloned is suitable for use in a GAL4/UAS expression system. Preferably, the expression vector comprises an UAS. By crossing the precursor insect which comprises the expression vector containing the UAS with a GAL4 line which is moreover tissue-specific, it is possible to control the expression of the human membrane transporter proteins in a simple and reliable manner.

In a further embodiment of the method, the transgenic insect is a Drosophila and the insect used in step c) comprising an expression system matched with the expression vector is a GAL4 Drosophila line.

Said embodiment is preferred because numerous GAL4 Drosophila lines are already known and are suitable for crossing within a UAS system. The GAL4 lines used can be, for example, P(fkh-Gal4), P(GawB)34B or P(GawB)C-765.

In a further aspect of the invention, said invention provides for the use of a transgenic insect for testing synthetic or natural compounds, especially drugs and active pharmacological ingredients.

With this embodiment, what can be provided by the transgenic insect is a novel and cost-effective in situ assay system which makes it possible to study interaction between human membrane transporter proteins and synthetic or natural compounds, especially drugs and active pharmacological ingredients. Thus, the transgenic insects can be used for pharmacological screenings. In this process, the interaction can be studied by various imaging techniques, for example by means of fluorescence spectroscopy.

In a further embodiment, the interaction can be studied by means of transporter-specific fluorescent tracer substrates. These can be injected into embryos of the transgenic insects which are transparent and express the human membrane transporter protein. Subsequently, the uptake of the fluorescent tracer substrate into the cells or tissues in which the human membrane transporter protein is specifically expressed can be observed, for example by fluorescence microscopy. What can be compared here is how the particular fluorescent tracer substrates behave in the absence or presence of the particular substances under study.

In this connection, substances which interact with an uptake transporter can lead to a reduced accumulation of the fluorescent tracer and hence to a reduced fluorescence within the cell. On the other hand, substances which interact with an efflux transporter can lead to an increased fluorescence in the particular tissue cells and a reduced fluorescent signal in the tissue lumen.

A particular advantage of this system is that up to 100 fly embryos can be injected and screened within 30 minutes. Furthermore, it is particularly advantageous that the model system is a very small insect embryo (0.5 mm), meaning that said system is suitable for automated analysis of the interaction of active ingredient with the membrane transporter proteins.

The new assay system, based on the transgenic insects, may be capable of replacing preexisting mouse models, the result being that a model which is ethically unobjectionable is created.

In a further embodiment of the use, the compound is tested for the interaction thereof with human membrane transporter proteins, the human membrane transporter protein being especially preferably selected from the group consisting of: OCT1, OCT2, OATP1B1, OATP1B3, OAT1, OAT3, MDR1, BSEP, BCRP, MATE1, MATE2 or a genetic variant transporters.

In a further aspect of the invention, said invention provides for the use of a transgenic insect for testing the function of human membrane transporter proteins, the human membrane transporter protein being especially preferably selected from the group consisting of: OCT1, OCT2, OATP1B1, OATP1B3, OAT1, OAT3, MDR1, BSEP, BCRP, MATE1, MATE2 or a genetic variant of these transporters.

It is evident that the above statements, which were made with respect to the membrane transporter proteins and the transgenic insect, also apply to their presently described use and to methods which are carried out according to the invention and as described below.

Besides the interaction with human membrane transporter proteins, it is also possible to study in the same model system the function of human membrane transporter proteins. The function of human membrane transporter proteins is preferably studied in situ. In this case, the particular substances to be studied are preferably injected into the insect embryos, these being introduced into gelatin for example. Subsequently, what are optionally prepared in a cryostat are thin sections (typically 20 μm thick) of the particular insect in a cryostat. Subsequently, a matrix substance is applied to the embryos or the sections thereof by pneumatic syringes. Said matrix consists of small organic molecules which absorb the laser energy introduced and ensure the desorption and ionization of the analytes. The particular substances to be studied can be directly measured with spatial resolution in the tissue of the embryos by the use of an atmospheric-pressure scanning microprobe matrix-assisted laser desorption ionization (SMALDI) ion source coupled with, for example, an orbital trap mass spectrometer. The images and data obtained can be used to determine the spatial distribution and amount of the substances to be studied in the particular tissues.

In a further aspect of the invention, said invention provides a method for testing a synthetic or natural compound to be studied with respect to the interaction thereof with human membrane transporter proteins, the human membrane transporter protein being especially preferably selected from the group consisting of: OCT1, OCT2, OATP1B1, OATP1B3, OAT1, OAT3, MDR1, BSEP, BCRP, MATE1, MATE2 or a genetic variant of these transporters, the method comprising the following successive steps:
a) providing embryos of a transgenic insect;
b) introducing a first tracer substance into the embryos and measuring the accumulation of the tracer substance in the cells or tissues of the embryos in which the human membrane transporter protein is specifically expressed; and/or measuring the accumulation of the tracer substance in the tissue-specific lumen of the embryos;
c) introducing a synthetic or natural compound to be studied into the embryos by injection into the embryos or incubation of the embryos in the dissolved compound;
d) measuring the accumulation of the tracer substance in the cells or tissues of the embryos in which the human membrane transporter protein is specifically expressed; and/or measuring the accumulation of the tracer substance in the tissue-specific lumens of the embryos; and
e) determining, on the basis of a reduced accumulation of the tracer substance or an unaltered accumulation of the tracer substance in the cells or tissues of the embryos in which the human membrane transporter protein is specifically expressed, whether the compound interacts with an uptake membrane transporter protein or not; and/or determining, on the basis of a reduced accumulation of the tracer substance or an unaltered accumulation of the tracer substance in the tissue-specific lumens of the embryos, whether the compound interacts with an efflux membrane transporter protein or not.

In a preferred embodiment of said method, the tissue-specific lumen of the embryos is selected from the salivary gland lumen, intestinal lumen, the Malpighian tubule lumen and the tracheal lumen of the embryos.

In a further preferred embodiment, measuring the accumulation of the tracer substance in the tissue-specific lumen of the embryos and/or determining, on the basis of a reduced accumulation of the tracer substance or an unaltered accumulation of the tracer substance in the tissue-specific lumens of the embryos, whether the compound interacts with an efflux membrane transporter protein or not is carried out with the aid of high-spatial-resolution imaging mass spectrometry or with the aid of fluorescence microscopy.

In a preferred embodiment, the compound to be studied is introduced in step b) within a period of approx. 5 min to 4 h, preferably approx. 60 min, after the simultaneous introduction of the tracer substance and a compound to be studied.

In a further aspect of the invention, said invention provides a method for directly testing the function of human membrane transporter proteins, the human membrane transporter protein being especially preferably selected from the group consisting of: OCT1, OCT2, OATP1B1, OATP1B3, OAT1, OAT3, MDR1, BSEP, BCRP, MATE1, MATE2 or a genetic variant of these transporters, the method comprising the following successive steps:
a) providing embryos of a transgenic insect;
b) introducing a synthetic or natural compound into the embryos by injection into the embryos or incubation of the embryos in the dissolved compound; and
c) measuring the presence and/or the amount of the compound introduced in step b) in the cells or tissues of the embryos in which the human membrane transporter protein is specifically expressed.

In this case, in a preferred embodiment of said method, measuring the accumulation of a synthetic or natural compound in the cells or tissues of the embryos in which the human membrane transporter protein is specifically expressed, especially in the cells of the salivary glands or other cell types of the embryos, and/or measuring the accumulation of the synthetic or natural compound in the cells or tissues of the embryos in which the human membrane transporter protein is specifically expressed, especially the salivary gland lumen or in the intestinal, Malpighian tubule or tracheal lumen of the embryos, is carried out with the aid of a high-spatial-resolution and/or high-resolution imaging mass spectrometry method or with the aid of fluorescence microscopy.

### ADVANTAGES OF THE INVENTION

The present invention is, in terms of its application, not limited to the details on creating and arranging the components that are presented in the description or illustrated in the drawings. The invention allows other embodiments and can be carried out in different ways. In addition, the manner of expression and terminology used here serve for descriptive purposes and is not to be regarded as limiting. In the present text, the use of “including”, “comprising” or “having”, “containing”, “encompassing” and variations thereof is intended to cover what is listed afterwards and also equivalents thereof and what is additional.

The present invention is further illustrated by the following examples, which are in no way to be understood as further limiting. The entire content of all information cited in the present application (including citations, granted patents, published patent applications and copending patent applications) is hereby expressly incorporated in the present text by reference. In the event of a conflict, the present description, including any definitions herein, takes precedence.

The figures are described in what follows.

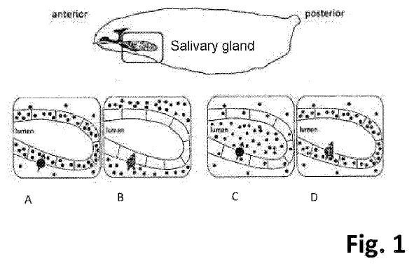

FIG. 1 shows a schematic representation of an insect embryo with the likewise schematically represented salivary gland as an exemplary specific tissue of an insect embryo (top), and also schematic representations of exemplary embodiments of the use of a transgenic insect according to the present invention (bottom), with an uptake membrane transporter protein (A, B) and with an efflux membrane transporter protein (C, D).

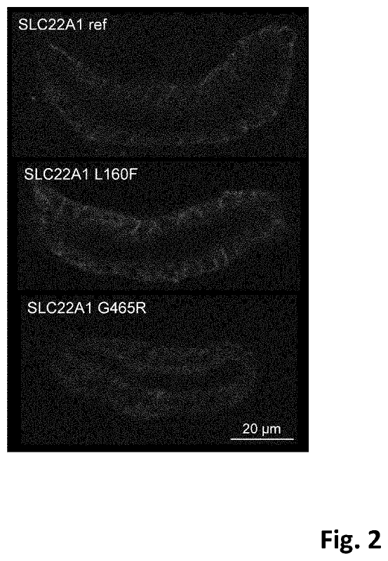

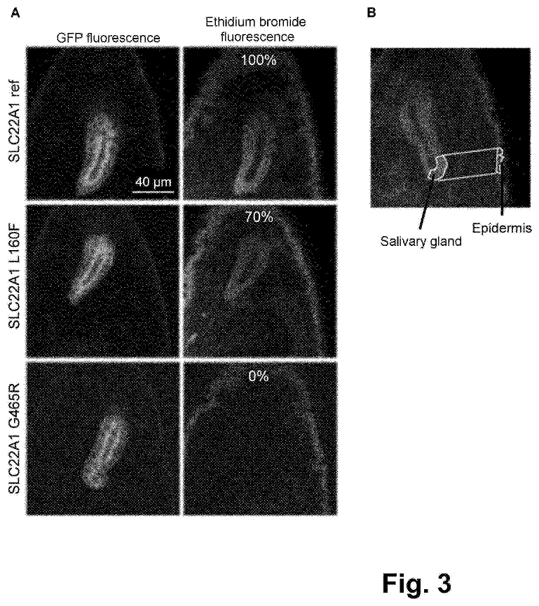

FIG. 2 shows immunofluorescence images of salivary glands of transgenic Drosophila embryos expressing either the reference sequence of the human SLC22A1/OCT1 protein (SLC22A1 ref) or genetic variants that occur in humans (e.g., SLC22A1 L160F; SLC22A1 G465R).

FIG. 3A shows the analysis of the uptake of fluorescent ethidium bromide, a transport substrate of OCT1, into the salivary gland of transgenic Drosophila embryos expressing either the reference sequence of the human SLC22A1/OCT1 protein (SLC22A1 ref) or genetic variants that occur in humans (e.g., SLC22A1 L160F; SLC22A1 G465R).

FIG. 3B shows the ethidium bromide uptake in the epidermis and the salivary gland.

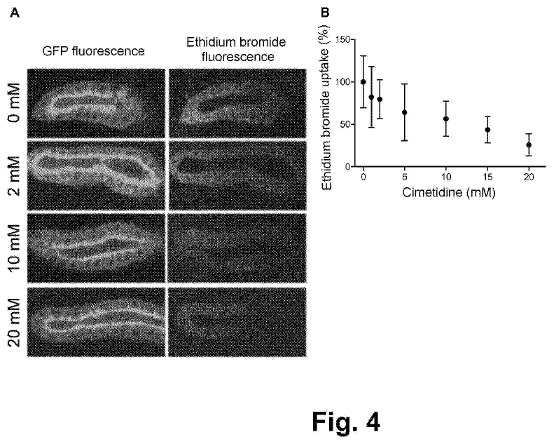

FIG. 4A shows the uptake of ethidium bromide into the salivary glands of transgenic Drosophila embryos expressing the reference sequence of the human SLC22A1/OCT1 protein.

FIG. 4B shows the inhibition of the uptake of ethidium bromide with increasing concentration of cimetidine.

FIG. 5 shows amino acid sequences of exemplary membrane transporter proteins which can be used in the context of the present invention, the following being shown here: OCT1 (SLC22A1) (A) (SEQ ID No. 1), OCT2 (SLC22A2) (B) (SEQ ID No. 2), OATP1B1 (SLCO1B1) (C) (SEQ ID No. 3), OATP1B3 (SLCO1B3) (D) (SEQ ID No. 4), OAT1 (SLC22A6) (E) (SEQ ID No. 5), OAT3 (SLC22A8) (F) (SEQ ID No. 6), MDR1 (ABCB1) (G) (SEQ ID No. 7), BSEP (ABCB11) (H) (SEQ ID No. 8), BCRP (ABCG2) (I) (SEQ ID No. 9), MATE1 (SLC47A1) (J) SEQ ID No. 10), MATE2 (SLC47A2) (K) (SEQ ID No. 11).

### EMBODIMENTS OF THE INVENTION

FIG. 1 depicts, in the top part, an insect embryo 10 (Drosophila), with depiction of the anterior on the left and the posterior on the right. Furthermore, the salivary gland 12 of the insect embryo is drawn in schematically. The salivary gland in the insect embryo consists of a monolayer of epithelial cells that form a lumen. On the basal side thereof, said cells come into contact with blood (hemolymph), and on the apical side, the cells secrete glycoproteins, which are only required during pupation for substrate adhesion by the animal, but not in the embryo or early larval stages.

The bottom part of FIG. 1 shows enlarged representations of salivary glands as exemplary specific tissue of two different embodiments of transgenic insects: in A and B, a human uptake membrane transporter protein is expressed in the transgenic insect, specifically in a tissue-specific manner, whereas in C and D, an efflux membrane transporter protein is expressed in a tissue-specific manner.

FIGS. 1 A, B show that the uptake membrane transporter protein is expressed in the basal membrane; the membrane transport protein takes up the tracer, which is supplied by means of injection (A). A simultaneous supply (e.g., injection) of a fluorescent tracer and a substance to be tested, for example a drug, inhibits the accumulation of the fluorescent tracer in the salivary gland (B) when the substance to be studied interacts with the uptake membrane transporter protein.

FIGS. 1 C, D show that the efflux membrane transporter protein is expressed in the apical membrane; said membrane transport protein transports the tracer, which must be initially taken up into the salivary gland cells, into the lumen of the salivary gland (C). A simultaneous supply (e.g., injection) of a fluorescent tracer and a substance to be tested, for example a drug, inhibits the accumulation of the fluorescent tracer in the lumen of the salivary gland and leads to an increased fluorescence in the salivary gland cells (D) when the substance to be studied interacts with the efflux membrane transporter protein.

FIG. 2 shows that the membrane transporter proteins OCT1/SLC22A1 reference sequence and its variants L160F and G465R are expressed in the salivary glands of Drosophila melanogaster embryos with the aid of the GAL4/UAS expression system. As is standard, the embryos were fixed for 20 min in 4% formaldehyde, at room temperature and with subsequent repeated washing in phosphate-buffered saline solution. With the aid of an OCT1/SLC22A1-specific antibody and a secondary antibody (Alexa Fluor 568 goat anti-mouse IgG, Invitrogen), the OCT1 proteins in the embryos were detected in standard methods. OCT1/SLC22A1 reference sequence and the variant L160F localize in the basal and lateral cell membrane, whereas the variant G465R can be found in the cytoplasm. What is essentially shown by this experiment is that human membrane transporter proteins are localized in fly embryos in the same way as in human cells.

FIG. 3 shows that the membrane transporter proteins OCT1/SLC22A1 reference sequence and its variants L160F and G465R are expressed in the salivary glands expressed of Drosophila melanogaster embryos with the aid of the GAL4/UAS expression system. The salivary glands are moreover characterized by the tissue-specific expression of GFP. These living embryos were injected with 0.5 μM ethidium bromide. The accumulation of ethidium bromide in the salivary glands was visualized by confocal laser scanning microscopy. The ratio of the signal in the salivary gland cells and in the epidermis cells, which naturally take up ethidium bromide and hence constitute the background, serves for the determination of the efficiency of uptake into the salivary gland cells. Differences in the uptake of ethidium bromide by the variants of, for example, OCT1 reference sequence can thus be established.

FIG. 4 shows that the membrane transporter protein OCT1/SLC22A1 reference sequence is expressed in the salivary glands of Drosophila melanogaster embryos with the aid of the GAL4/UAS expression system. The salivary glands are moreover characterized by the tissue-specific expression of GFP. Ethidium bromide and cimetidine were simultaneously injected into the ventral side of the embryos. The inhibition of the uptake of ethidium bromide by cimetidine is concentration-dependent. The effect of cimetidine is the same in insects as in human cells.

As already mentioned above, FIG. 5 shows amino acid sequences of exemplary membrane transport proteins which can be used in the context of the present invention. The amino acid sequences of the following membrane transport proteins are shown here by way of example: OCT1 (SLC22A1) (A), OCT2 (SLC22A2) (B), OATP1B1 (SLCO1B1) (C), OATP1B3 (SLCO1B3) (D), OAT1 (SLC22A6) (E), OAT3 (SLC22A8) (F), MDR1 (ABCB1) (G), BSEP (ABCB11) (H), BCRP (ABCG2) (I), MATE1 (SLC47A1) (J), MATE2 (SLC47A2) (K).

1. A transgenic insect, comprising:
a genome including at least one first exogenous DNA sequence encoding a human membrane transporter protein, the expression of the first exogenous DNA sequence leading to a functional human membrane transporter protein in the transgenic insect.

2. The transgenic insect as claimed in claim 1, wherein the expression of the first exogenous DNA sequence of the human membrane transporter protein in the insect is one or more of salivary gland tissue-specific, intestinal system tissue-specific, tracheal tissue-specific, and Malpighian tissue-specific.

3. The transgenic insect as claimed in claim 1, wherein the transgenic insect is a fruit fly belonging to the genus Drosophila.

4. The transgenic insect as claimed in claim 1, wherein the human membrane transporter protein is a human uptake transporter protein or a human efflux transporter protein.

5. The transgenic insect as claimed in claim 1, wherein the expression of the first exogenous DNA sequence encoding a human membrane transporter protein is under the control of one or more of a salivary gland tissue-specific GAL4/UAS, an intestinal system tissue-specific GAL4/UAS, a tracheal tissue-specific GAL4/UAS, and Malpighian tissue-specific GAL4/UAS expression system.

6. The transgenic insect as claimed in claim 1, wherein the genome thereof comprises at least one second exogenous DNA sequence encoding including at least one fluorescent protein selected from the group consisting of GFP, CFP, YFP, mCherry, dsRed, and variants thereof.

7. The transgenic insect as claimed in claim 1, wherein the transgenic insect is a transgenic Drosophila, the genome further including:
a vector wherein the human membrane transporter protein is an uptake transporter protein or an efflux transporter protein selected from the group consisting of: OCT1, OCT2, OATP1B1, OATP1B3, OAT1, OAT3, MDR1, BSEP, BCRP, MATE1, MATE2, and genetic variants thereof.

8. A method for generating a transgenic insect, comprising:
subcloning a first exogenous DNA sequence encoding a human membrane transporter protein into an expression vector to obtain a vector comprising the first exogenous DNA sequence;
introducing the vector comprising the first exogenous DNA sequence into an insect to obtain a stable strain of a transgenic precursor insect; and
crossing the transgenic precursor insect with an insect comprising an expression system matched with the expression vector to obtain a transgenic insect with a functional human membrane transporter protein.

9. The method as claimed in claim 8, wherein the expression system is GAL4/UAS.

10. The method as claimed in claim 8, wherein the transgenic insect is a Drosophila and the insect comprising the expression system matched with the expression vector is a GAL4 Drosophila line.

11. The method as claimed in claim 14, wherein introducing a selected one of a synthetic compound to be studied and a or natural compound to be studied comprises:
introducing a selected one of a synthetic drug or active pharmacological ingredient to be studied and a natural drug or active pharmacological ingredient to be studied.

12. (canceled)

13. The transgenic insect as claimed in claim 1, wherein the human membrane transporter protein is selected from the group consisting of: OCT1, OCT2, OATP1B1, OATP1B3, OAT1, OAT3, MDR1, BSEP, BCRP, MATE1, MATE2 and genetic variants thereof.

14. A method for testing a synthetic or natural compound to be studied with respect to the interaction thereof with a human membrane transporter protein, the method comprising:
selecting a human membrane transporter protein from the group consisting of: OCT1, OCT2, OATP1B1, OATP1B3, OAT1, OAT3, MDR1, BSEP, BCRP, MATE1, MATE2, and genetic variants thereof;
providing at least one embryo of a transgenic insect with the selected human membrane transporter protein;
introducing a tracer substance into the at least one embryo;
initially measuring an accumulation of the tracer substance in at least one of 1) cells or tissues of the at least one embryo in which the selected human membrane transporter protein is specifically expressed, and 2) a tissue-specific lumen of the at least one embryo;
introducing a selected one of a synthetic compound to be studied and a natural compound to be studied into the at least one embryo by at least one of injection into the at least one embryo and incubation of the embryos in a dissolved form of the selected compound;
subsequently measuring, after introducing the selected compound, an accumulation of the tracer substance in at least one of 1) the cells or tissues of the at least one embryo in which the human membrane transporter protein is specifically expressed, and 2) the tissue-specific lumens of the at least one embryo, with the aid of a selected one of a high-spatial-resolution imaging mass spectrometry method, and a fluorescence microscopy; and
determining, with the aid of the selected one of a high-spatial-resolution imaging mass spectrometry method and fluorescence microscopy, that
the selected compound interacts with an uptake membrane transporter protein when a reduced accumulation of the tracer substance is measured, in the cells or tissues of the at least one embryo in which the human membrane transporter protein is specifically expressed, in the subsequent measurement compared to the initial measurement,
the selected compound does not interact with an uptake membrane transporter protein when an unaltered accumulation of the tracer substance is measured, in the cells or tissues of the at least one embryo in which the human membrane transporter protein is specifically expressed, in the subsequent measurement compared to the initial measurement,
the selected compound interacts with an efflux membrane transporter protein when a reduced accumulation of the tracer substance is measured, in the tissue-specific lumens of the at least one embryo, in the subsequent measurement compared to the initial measurement, and
the selected compound does not interact with an efflux membrane transporter protein when an unaltered accumulation of the tracer substance is measured, in the tissue-specific lumens of the at least one embryo, in the subsequent measurement compared to the initial measurement.

15. The method as claimed in claim 14, wherein the selected compound to be studied is introduced within a period of about 5 minutes to 4 hours after the introduction of the tracer substance.

16. (canceled)

17. The transgenic insect as claimed in claim 1, wherein the human membrane transporter protein is a human membrane transporter protein selected from the group consisting of: OCT1, OCT2, OATP1B1, OATP1B3, OAT1, OAT3, MDR1, BSEP, BCRP, MATE1, MATE2 or a genetic variant of these transporters.

18. The method as claimed in claim 14, wherein the selected compound to be studied is introduced within a period of about 60 min, after the introduction of the tracer substance.


SEQUENCE LISTING (11 sequences)

SEQ ID NO: 1 - protein, 554 aa, Homo sapiens
MPTVDDILEQVGESGWFQKQAFLILCLLSAAFAPICVGIVFLGFTPDHHCQSPGVAELSQRCGWSPAEELNYTVPGLGPAGEAFLGQCRRYEVDWNQSALSCVDPLASLATNRSHLPLGPCQDGWVYDTPGSSIVTEFNLVCADSWKLDLFQSCLNAGFLFGSLGVGYFADRFGRKLCLLGTVLVNAVSGVLMAFSPNYMSMLLFRLLQGLVSKGNWMAGYTLITEFVGSGSRRTVAIMYQMAFTVGLVALTGLAYALPHWRWLQLAVSLPTFLFLLYYWCVPESPRWLLSQKRNTEAIKIMDHIAQKNGKLPPADLKMLSLEEDVTEKLSPSFADLFRTPRLRKRTFILMYLWFTDSVLYQGLILHMGATSGNLYLDFLYSALVEIPGAFIALITIDRVGRIYPMAMSNLLAGAACLVMIFISPDLHWLNIIIMCVGRMGITIAIQMICLVNAELYPTFVRNLGVMVCSSLCDIGGIITPFIVFRLREVWQALPLILFAVLGLLAAGVTLLLPETKGVALPETMKDAENLGRKAKPKENTIYLKVQTSEPSGT

SEQ ID NO: 2 - protein, 555 aa, Homo sapiens
MPTTVDDVLEHGGEFHFFQKQMFFLLALLSATFAPIYVGIVFLGFTPDHRCRSPGVAELSLRCGWSPAEELNYTVPGPGPAGEASPRQCRRYEVDWNQSTFDCVDPLASLDTNRSRLPLGPCRDGWVYETPGSSIVTEFNLVCANSWMLDLFQSSVNVGFFIGSMSIGYIADRFGRKLCLLTTVLINAAAGVLMAISPTYTWMLIFRLIQGLVSKAGWLIGYILITEFVGRRYRRTVGIFYQVAYTVGLLVLAGVAYALPHWRWLQFTVALPNFFFLLYYWCIPESPRWLISQNKNAEAMRIIKHIAKKNGKSLPASLQRLRLEEETGKKLNPSFLDLVRTPQIRKHTMILMYNWFTSSVLYQGLIMHMGLAGDNIYLDFFYSALVEFPAAFMIILTIDRIGRRYPWAASNMVAGAACLASVFIPGDLQWLKIIISCLGRMGITMAYEIVCLVNAELYPTFIRNLGVHICSSMCDIGGIITPFLVYRLTNIWLELPLMVFGVLGLVAGGLVLLLPETKGKALPETIEEAENMQRPRKNKEKMIYLQVQKLDIPLN

SEQ ID NO: 3 - protein, 691 aa, Homo sapiens
MDQNQHLNKTAEAQPSENKKTRYCNGLKMFLAALSLSFIAKTLGAIIMKSSIIHIERRFEISSSLVGFIDGSFEIGNLLVIVFVSYFGSKLHRPKLIGIGCFIMGIGGVLTALPHFFMGYYRYSKETNINSSENSTSTLSTCLINQILSLNRASPEIVGKGCLKESGSYMWIYVFMGNMLRGIGETPIVPLGLSYIDDFAKEGHSSLYLGILNAIAMIGPIIGFTLGSLFSKMYVDIGYVDLSTIRITPTDSRWVGAWWLNFLVSGLFSIISSIPFFFLPQTPNKPQKERKASLSLHVLETNDEKDQTANLTNQGKNITKNVTGFFQSFKSILTNPLYVMFVLLTLLQVSSYIGAFTYVFKYVEQQYGQPSSKANILLGVITIPIFASGMFLGGYIIKKFKLNTVGIAKFSCFTAVMSLSFYLLYFFILCENKSVAGLTMTYDGNNPVTSHRDVPLSYCNSDCNCDESQWEPVCGNNGITYISPCLAGCKSSSGNKKPIVFYNCSCLEVTGLQNRNYSAHLGECPRDDACTRKFYFFVAIQVLNLFFSALGGTSHVMLIVKIVQPELKSLALGFHSMVIRALGGILAPIYFGALIDTTCIKWSTNNCGTRGSCRTYNSTSFSRVYLGLSSMLRVSSLVLYIILIYAMKKKYQEKDINASENGSVMDEANLESLNKNKHFVPSAGADSETHC

SEQ ID NO: 4 - protein, 702 aa, Homo sapiens
MDQHQHLNKTAESASSEKKKTRRCNGFKMFLAALSFSYIAKALGGIIMKISITQIERRFDISSSLAGLIDGSFEIGNLLVIVFVSYFGSKLHRPKLIGIGCLLMGTGSILTSLPHFFMGYYRYSKETHINPSENSTSSLSTCLINQTLSFNGTSPEIVEKDCVKESGSHMWIYVFMGNMLRGIGETPIVPLGISYIDDFAKEGHSSLYLGSLNAIGMIGPVIGFALGSLFAKMYVDIGYVDLSTIRITPKDSRWVGAWWLGFLVSGLFSIISSIPFFFLPKNPNKPQKERKISLSLHVLKTNDDRNQTANLTNQGKNVTKNVTGFFQSLKSILTNPLYVIFLLLTLLQVSSFIGSFTYVFKYMEQQYGQSASHANFLLGIITIPTVATGMFLGGFIIKKFKLSLVGIAKFSFLTSMISFLFQLLYFPLICESKSVAGLTLTYDGNNSVASHVDVPLSYCNSECNCDESQWEPVCGNNGITYLSPCLAGCKSSSGIKKHTVFYNCSCVEVTGLQNRNYSAHLGECPRDNTCTRKFFIYVAIQVINSLFSATGGTTFILLTVKIVQPELKALAMGFQSMVIRTLGGILAPIYFGALIDKTCMKWSTNSCGAQGACRIYNSVFFGRVYLGLSIALRFPALVLYIVFIFAMKKKFQGKDTKASDNERKVMDEANLEFLNNGEHFVPSAGTDSKTCNLDMQDNAAAN

SEQ ID NO: 5 - protein, 563 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRWAPTQKEAGIYPRKGKQTRQQQEHQKYMVPLQASAQEKNGL

SEQ ID NO: 6 - protein, 542 aa, Homo sapiens
MTFSEILDRVGSMGHFQFLHVAILGLPILNMANHNLLQIFTAATPVHHCRPPHNASTGPWVLPMGPNGKPERCLRFVHPPNASLPNDTQRAMEPCLDGWVYNSTKDSIVTEWDLVCNSNKLKEMAQSIFMAGILIGGLVLGDLSDRFGRRPILTCSYLLLAASGSGAAFSPTFPIYMVFRFLCGFGISGITLSTVILNVEWVPTRMRAIMSTALGYCYTFGQFILPGLAYAIPQWRWLQLTVSIPFFVFFLSSWWTPESIRWLVLSGKSSKALKILRRVAVFNGKKEEGERLSLEELKLNLQKEISLAKAKYTASDLFRIPMLRRMTFCLSLAWFATGFAYYSLAMGVEEFGVNLYILQIIFGGVDVPAKFITILSLSYLGRHTTQAAALLLAGGAILALTFVPLDLQTVRTVLAVFGKGCLSSSFSCLFLYTSELYPTVIRQTGMGVSNLWTRVGSMVSPLVKITGEVQPFIPNIIYGITALLGGSAALFLPETLNQPLPETIEDLENWSLRAKKPKQEPEVEKASQRIPLQPHGPGLGSS

SEQ ID NO: 7 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 8 - protein, 1321 aa, Homo sapiens
MSDSVILRSIKKFGEENDGFESDKSYNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 9 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 10 - protein, 570 aa, Homo sapiens
MEAPEEPAPVRGGPEATLEVRGSRCLRLSAFREELRALLVLAGPAFLVQLMVFLISFISSVFCGHLGKLELDAVTLAIAVINVTGVSVGFGLSSACDTLISQTYGSQNLKHVGVILQRSALVLLLCCFPCWALFLNTQHILLLFRQDPDVSRLTQTYVTIFIPALPATFLYMLQVKYLLNQGIVLPQIVTGVAANLVNALANYLFLHQLHLGVIGSALANLISQYTLALLLFLYILGKKLHQATWGGWSLECLQDWASFLRLAIPSMLMLCMEWWAYEVGSFLSGILGMVELGAQSIVYELAIIVYMVPAGFSVAASVRVGNALGAGDMEQARKSSTVSLLITVLFAVAFSVLLLSCKDHVGYIFTTDRDIINLVAQVVPIYAVSHLFEALACTSGGVLRGSGNQKVGAIVNTIGYYVVGLPIGIALMFATTLGVMGLWSGIIICTVFQAVCFLGFIIQLNWKKACQQAQVHANLKVNNVPRSGNSALPQDPLHPGCPENLEGILTNDVGKTGEPQSDQQMRQEEPLPEHPQDGAKLSRKQLVLRRGLLLLGVFLILLVGILVRFYVRIQ

SEQ ID NO: 11 - protein, 602 aa, Homo sapiens
MDSLQDTVALDHGGCCPALSRLVPRGFGTEMWTLFALSGPLFLFQVLTFMIYIVSTVFCGHLGKVELASVTLAVAFVNVCGVSVGVGLSSACDTLMSQSFGSPNKKHVGVILQRGALVLLLCCLPCWALFLNTQHILLLFRQDPDVSRLTQDYVMIFIPGLPVIFLYNLLAKYLQNQGWLKGQEEESPFQTPGLSILHPSHSHLSRASFHLFQKITWPQVLSGVVGNCVNGVANYALVSVLNLGVRGSAYANIISQFAQTVFLLLYIVLKKLHLETWAGWSSQCLQDWGPFFSLAVPSMLMICVEWWAYEIGSFLMGLLSVVDLSAQAVIYEVATVTYMIPLGLSIGVCVRVGMALGAADTVQAKRSAVSGVLSIVGISLVLGTLISILKNQLGHIFTNDEDVIALVSQVLPVYSVFHVFEAICCVYGGVLRGTGKQAFGAAVNAITYYIIGLPLGILLTFVVRMRIMGLWLGMLACVFLATAAFVAYTARLDWKLAAEEAKKHSGRQQQQRAESTATRPGPEKAVLSSVATGSSPGITLTTYSRSECHVDFFRTPEEAHALSAPTSRLSVKQLVIRRGAALGAASATLMVGLTVRILATRH